(19) United States
(12) Patent Application Publication
(10) Pub. No.: US 2015/0105812 A1
(43) Pub. Date: Apr. 16, 2015

(54) PACEMAKER CONTACT SCREW REMOVAL TOOL

(71) Applicant: Brian James Cutler, Rowland Heights, CA (US)

(72) Inventor: Brian James Cutler, Rowland Heights, CA (US)

(21) Appl. No.: 14/511,724

(22) Filed: Oct. 10, 2014

Related U.S. Application Data
(60) Provisional application No. 61/889,822, filed on Oct. 11, 2013.

(51) Int. Cl. A61B 17/3205; A61B 19/00; A61N 1/375

(52) U.S. Cl. CPC A61B 17/3205; A61N 1/3752; A61B 19/30; A61B 2017/00831
USPC 606/180

(57) ABSTRACT

A tool for removing debris from sockets containing screws of electrical conductors of medical pacemakers, to allow engagement of screw heads for unthreading. The tool includes a body, with a screw driving blade projecting from one end and a cutting blade of diameter similar to that of the screw driving blade projecting from an opposed end. Optionally, the body encloses torque limiting components limiting torque applied to the screw driving blade. The screw driving blade may be polygonal. The cutting blade may be a fluted drill bit. Where the torque limiting components are not provided, the body may bear external splines, with the screw driving blade and cutting blade being monolithic with the body.

### CROSS-REFERENCE TO RELATED APPLICATION

This application claims the benefit of U.S. Provisional Application Ser. No. 61/889,822, filed Oct. 11, 2013, which is hereby explicitly incorporated herein by reference.

### FIELD OF THE DISCLOSURE

The present disclosure relates to drive tools, and more particularly, to a drive tool for providing operable access to and removing screws from, for example, medical pacemakers.

### BACKGROUND

Electrical contacts in medical pacemakers must from time to time be removed to permit servicing or replacement of the medical pacemakers. These electrical contacts, their conductors, or conductive terminals, are held in place by screws which must be loosened to disconnect the contacts. The screws occupy sockets into which a screwdriver must reach. However, in the body environment, the screws may become covered by body tissue debris. This matter may interfere with ability of screwdrivers to successfully engage and drive the screws. For example, the tip of the screwdriver may become stripped as a cardiologist attempts to remove these screws if the socket is clogged by tissue debris.

### SUMMARY

The inventive tool addresses the above stated situation by providing a tool incorporating both a screw driver element and also an auger type blade for removing tissue debris which obstructs access to the screw head. Optionally, the tool may further incorporate a torque limiting feature which prevents overtightening of the screw(s).

To these ends, the novel tool includes a body, a screw driving blade projecting from one end of the body, and an auger type cutting blade projecting from an opposed end of the body. The body contains within torque limiting components, where provided, acting on the screw driving blade.

It is an object to provide improved elements and arrangements thereof by apparatus for the purposes described which is inexpensive, dependable, and fully effective in accomplishing its intended purposes.

These and other objects will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is an exploded perspective view of a tool for gaining access to and driving screws for pacemakers, according to at least one aspect of the disclosure;

FIG. 2 is a side view of the tool of FIG. 1, showing the tool assembled, and further showing caps exploded from the tool; and

FIG. 3 is a side view, partially in cross section, of a tool for gaining access to and driving screws for pacemakers, according to at least one further aspect of the disclosure.

FIG. 4 is an enlarged environmental cross sectional detail view of a cutting bit of the tool of FIG. 1, shown entering a screw socket containing a screw.

### DETAILED DESCRIPTION

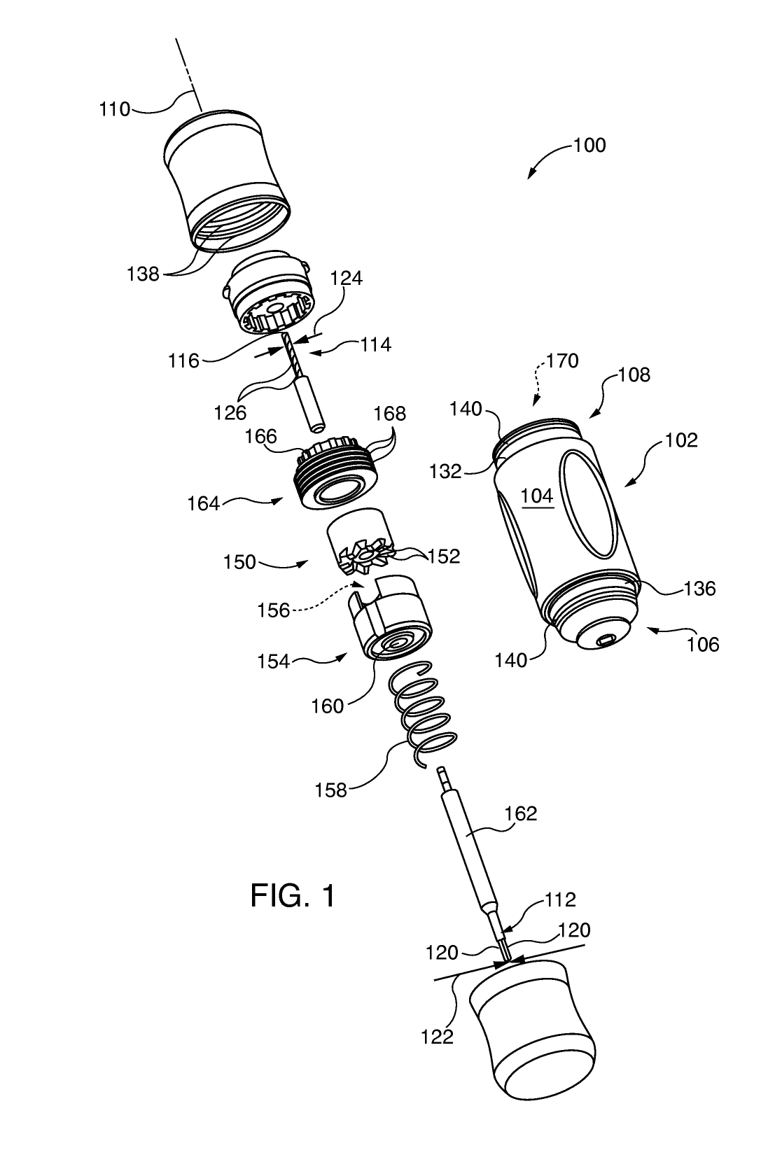

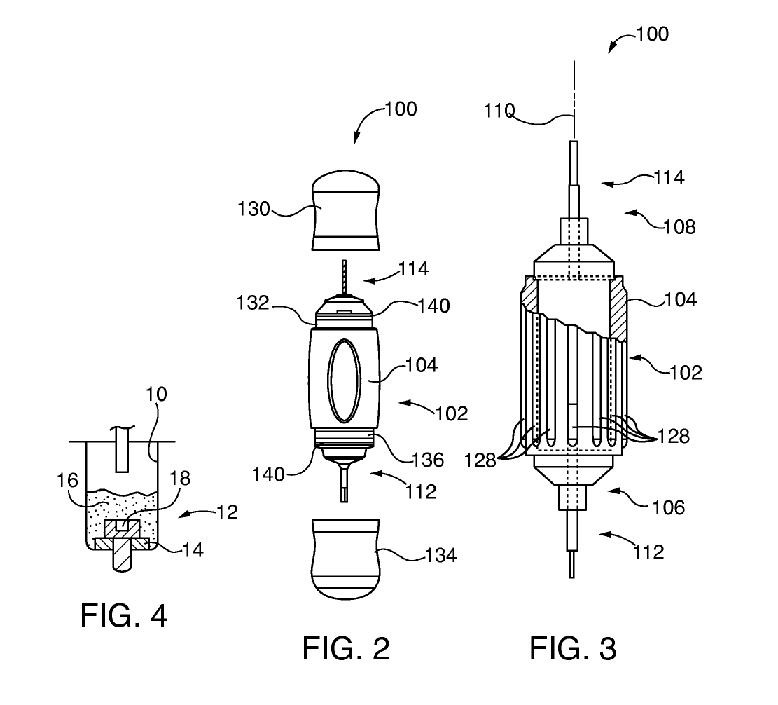

Referring first to FIGS. 1 and 2, according to at least one aspect of the invention, there is shown a tool 100 for gaining access to and driving screws for pacemakers (not shown). The tool 100 comprises a body 102 including an outer enclosure 104, a proximal end 106, a distal end 108, and an axis of rotation 110. The tool 100 also comprises a screw driving blade 112 projecting from body 102 along axis of rotation 110, from proximal end 106, and a cutting bit 114 supported by body 102 along axis of rotation 110, cutting bit 114 projecting from body 102 from distal end 108. Cutting bit 114 is fluted and has an exposed cutting tip 116 projecting from body 102. Screw driving blade 112, cutting bit 114, and orientation of these on body 102 along axis of rotation 110 facilitate access to and removal of screws for pacemakers.

Body 102 is a structural member holding functional components of tool 100 in operable positions aligned along axis of rotation 110. Body 102 encloses and protects optional torque limiting components, which will be further described hereinafter.

Optionally, cutting bit 114 is fixed to body 102. This may be accomplished by overmolding body 102 with respect to cutting blade 114, by bonding cutting blade 114 to body 102, by crimping cutting blade 114 to body 102, or in any other suitable way.

Referring also to FIG. 4, where a socket 10 contains a screw 12 fastening an electrical conductor or terminal 14 of the latter in place within a medical pacemaker (not shown in its entirety), debris 16 from body tissues may come to clog socket 10. Cutting bit 114 bores through debris 16, including most of the debris 16 occupying a drive hole 18 of screw 12, and evacuates debris 16 from screw 12 and socket 10.

In at least one implementation of the disclosure, cutting bit 114 has a biologically inert external layer. Cutting bit 114 having a biologically inert external layer avoids metallic or other chemical contamination of the body. In a further implementation of the disclosure, the biologically inert external layer may comprise titanium. Titanium is a durable, biologically inert material highly suitable for cutting bit 114. A biologically inert layer comprising titanium may be achieved by coating cutting bit 114 with titanium or a titanium compound, such as titanium nitride. It would also be possible to fabricate all or much of cutting bit 114 from titanium or an alloy thereof.

In at least one implementation of the disclosure, cutting tip 116 is blunt ended. This configuration enables cutting tip 116 to enter drive hole 18 and remove most of the debris 16 where the bottom of drive hole 18 has a flat floor.

In at least one implementation of the disclosure, screw driving blade 112 is polygonal in cross section, has a cross sectional diameter 122 between two opposed sides 120 of screw driving blade 112. Cutting bit 114 has a diameter 124 equal to a dimension between two opposed sides 120 of screw driving blade 112. The dimension between two opposed sides 120 of screw driving blade 112 is seen as cross sectional diameter 122 in FIG. 1. This relationship causes cutting bit 114 to remove most of the debris 16 from drive hole 18 of screw 12, thereby enabling screw driving blade 112 to engage drive hole 18 without rounding corners or apices of screw driving blade 112.

In at least one implementation of the disclosure, cutting bit 114 has two flutes 126. Two flutes 126 increase and may maximize debris evacuation capacity of cutting bit 114 as the latter is rotated to remove debris 16.

In at least one implementation of the disclosure, tool 100 further comprises a first cap 130 configured to cover cutting bit 114 and retainably engage body 102. First cap 130 slips over and engages by friction a surface 132 of body 102. First cap 130 protects people and objects from damage due to contact with exposed cutting edges of cutting bit 114, and protects cutting bit 114 from damage due to contact with an external object (not shown).

In at least one implementation of the disclosure, tool 100 further comprises a second cap 134 configured to cover screw driving blade 112 and retainably engage body 102. Second cap 134 slips over and engages by friction a surface 136 of body 102. Second cap 134 protects people and objects from damage due to contact with screw driving blade 112.

First and second caps 130, 134 may be fabricated from a slightly flexible material such as a synthetic polymer or natural rubber. First and second caps 130, 134 may comprise internal ribs 138 (FIG. 1) which engage corresponding ribs 140 of body 102 to promote secure retention of first and second caps 130, 134.

Referring specifically to FIG. 1, in at least one implementation of the disclosure, tool 100 comprises a torque limiting feature operable to limit torque imparted to screw driving blade 112 when rotating body 102. The torque limiting components act on screw driving blade 112 to torque a screw such as screw 12 of FIG. 4 to a specified torque value, without exceeding that torque value. Hence tool 100 can be expeditiously used without obliging the user to monitor or carefully limit torques imposed on the item driven by tool 100.

Torque limiting components for screw driving blade 112 include a first member 150 having downwardly projecting teeth 152 (downwardly projecting as seen in FIG. 1), each tooth 152 including a ramp arranged at a small angle to a hypothetical plane perpendicular to rotational axis 110. The small angle may be for example five to thirty degrees from the hypothetical plane.

First member 150 has an opposed second member 154 bearing teeth 156. Second member 154 is fixed to body 102. An aperture 160 in second member 154 is configured to cooperate closely yet slidably with the outer surface of shaft 162 of screw driving blade 112. For example, both the outer surface of shaft 162 and the inner surface of aperture 160 may be hexagonal.

Although not visible in the view of FIG. 1, teeth 156 are arranged in a complementary manner, interfitting with teeth 152 but being opposite in pitch. When first and second members 150, 154 are mutually rotated about rotational axis 110 in one direction, initially, second member 154 rotates first member 150. As resistance from an object driven by 112 increases, first and second members 150, 154 will mutually repel or displace one another as the opposed ramps of teeth 152, 156 slide across one another. Rotation of first member 150 continues until first and second members 150, 154 are mutually displaced to the point that the apices of teeth 152, 156 slide past each other. A spring 158 adjustably varies resistance to mutual displacement of first and second members 150, 154, thereby varying the maximum torque which can be transmitted to second member 154 from first member 150.

A threaded plug 164 is adjusted by a wrench (not shown) which cooperates with castellation 166 to adjust resistance of spring 158. Threads 168 of threaded plug 164 engage threads 170 (concealed from view in FIG. 1) of body 102. The maximum torque value is calibrated by determining position of threaded plug 164 along body 102 prior to fixing threaded plug 164 to body 102. Threaded plug 164 may engage body 102 by glue, by distortion to threads 168 or 170, or in any other suitable way.

Referring now to FIG. 3, there is shown a tool 100 for gaining access to and driving screws for pacemakers. Tool 100 comprises a body 102 including an outer enclosure 104, a proximal end 106, a distal end 108, and an axis of rotation 110. The tool 100 also comprises a screw driving blade 112 projecting from body 102 along axis of rotation 110, from proximal end 106, and a cutting bit 114 supported by body 102 along axis of rotation 110, cutting bit 114 projecting from body 102 from distal end 108. In the implementation of FIG. 3, screw driving blade 112 is fixed to body 102. Body 102 is solid, screw driving blade 112 and cutting bit 114 embedded within body 102. Body 102 includes external splines 128. External splines 128 not only facilitate secure manual grasp of body 102, but also enable tool 100 to cooperate with a torque wrench (not shown) having a socket cooperating with body 102. This enables an inexpensive version of tool 100 to be realized.

Tool 100 has been described for use in the environment of medical pacemakers only for convenience of understanding in terms of applying tool 100 to an environment having recognizable characteristics. It should be realized that tool 100 is not limited to this environment, and may be utilized in any environment where it is desired to remove debris or other material, such as sealing materials, to gain access to and drive the head of a fastener or other object.

Further, as a non-limiting example, the cutting bit 114 may be a separate tool onto itself. The cutting bit may be detached from the torque tool 100 and attached to an accompanied handle supporting the cutting bit. As such, the cutting bit may be used as a separate tool to clean out the screw socket.

Unless otherwise indicated, the terms “first”, “second”, etc., are used herein merely as labels, and are not intended to impose ordinal, positional, or hierarchical requirements on the times to which these terms refer. Moreover, reference to, e.g., a “second” item does not either require or preclude the existence of, e.g., a “first” or lower-numbered item, and/or, e.g., a “third” or higher-numbered item.

It should be understood that the various examples of the apparatus(es) disclosed herein may include any of the components, features, and functionalities of any of the other examples of the apparatus(es) disclosed herein in any feasible combination, and all of such possibilities are intended to be within the spirit and scope of the present disclosure. Many modifications of examples set forth herein will come to mind to one skilled in the art to which the present disclosure pertains having the benefit of the teachings presented in the foregoing descriptions and the associated drawings.

Therefore, it is to be understood that the present disclosure is not to be limited to the specific examples presented and that modifications and other examples are intended to be included within the scope of the appended claims. Moreover, although the foregoing description and the associated drawings describe examples of the present disclosure in the context of certain illustrative combinations of elements and/or functions, it should be appreciated that different combinations of elements and/or functions may be provided by alternative implementations without departing from the scope of the appended claims.

I claim:

1. A tool for gaining access to and driving screws for pacemakers, comprising:
a body including an outer enclosure, a proximal end, a distal end, and an axis of rotation;
a screw driving blade projecting from the body along the axis of rotation from the proximal end; and
a cutting bit supported by the body along the axis of rotation from the distal end, wherein the cutting bit is fluted and has an exposed cutting tip projecting from the body.

2. The tool of claim 1, wherein the cutting tip is blunt ended.

3. The tool of claim 1, wherein the cutting bit has two flutes.

4. The tool of claim 1, wherein:
the screw driving blade is polygonal in cross section and has a cross sectional diameter between two opposed sides of the screw driving blade; and
the cutting bit has a diameter equal to a dimension between the two opposed sides of the screw driving blade.

5. The tool of claim 1, wherein the cutting bit has a biologically inert external layer

6. The tool of claim 5, wherein the biologically inert external layer comprises titanium.

7. The tool of claim 1, further comprising a torque limiting feature operable to limit torque imparted to the screw driving blade when rotating the body.

8. The tool of claim 1, wherein the body includes external splines.

9. The tool of claim 1, wherein the cutting bit is fixed to the body.

10. The tool of claim 1, wherein the screw driving blade is fixed to the body.

11. The tool of claim 1, further comprising a first cap configured to cover the cutting bit and retainably engage the body.

12. The tool of claim 1, further comprising a second cap configured to cover the screw driving blade and retainably engage the body.
